(19) United States
(12) Patent Application Publication
(10) Pub. No.: US 2007/0036367 A1
(43) Pub. Date: Feb. 15, 2007

(54) Apparatus and method of reducing noise of earphones, noise reducing earphones, and a portable audio reproducing apparatus having the same

(75) Inventor: Byeong-seob Ko, Suwon-si (KR)

Correspondence Address: STANZIONE & KIM, LLP, 919 18TH STREET, N.W., SUITE 440, WASHINGTON, DC, 20006, US

(73) Assignee: Samsung Electronics Co., Ltd., Suwon-si (KR)

(21) Appl. No.: 11/541,660

(22) Filed: Oct. 3, 2006

(30) Foreign Application Priority Data
Jan. 14, 2005 (KR) 2006-4174

(51) Int. Cl. A61F 11/06; G10K 11/16; H03B 29/00

(52) U.S. Cl. 381/71.1

(57) ABSTRACT

An apparatus and method of reducing noise in a portable audio reproducing apparatus using earphones. The apparatus includes an earphone unit having a speaker unit and a microphone to output an audio signal and to generate anti-noise with respect to external noise, and a circuit unit to compensate a frequency characteristic of the anti-noise generated by the microphone of the earphone unit, to add the anti-noise to an input audio signal, and to remove background noise using the anti-noise by outputting the audio signal having the anti-noise to the speaker unit.

### CROSS-REFERENCE TO RELATED APPLICATIONS

This application claims the benefit of Korean Patent Application No. 10-2006-0004174, filed on Jan. 14, 2006, in the Korean Intellectual Property Office, the disclosure of which is incorporated herein in its entirety by reference.

### BACKGROUND OF THE INVENTION

1. Field of the Invention

The present general inventive concept relates to a portable audio reproducing apparatus, and more particularly, to an apparatus and method of reducing noise in a portable audio reproducing apparatus using earphones.

2. Description of the Related Art

With the development of related industries, audio reproducing apparatuses capable of reproducing sound, such as music and voice, are becoming more portable. These portable audio reproducing apparatuses allow music to be listened to or languages to be studied irrespective of places, such as in a subway train, on a bus, or on the road. Examples of these portable audio reproducing apparatuses include an MP3 player, a media player, a mini cassette, and a compact disc player.

Generally, the portable audio reproducing apparatus is an apparatus that reproduces data recorded in a memory or on a recording medium, such as a CD, or reproduces a broadcasting signal received from a broadcasting station. An audio signal reproduced by the portable audio reproducing apparatus is amplified through an amplifier and is output to earphones. The earphones output the unilaterally amplified audio signal through speaker units. However, due to noise flowing in from an external environment, the earphones of the portable audio reproducing apparatus degrade the quality of sound.

Accordingly, in order to maintain a sound volume considering the external noise, users would need to increase a sound pressure of the portable audio reproducing apparatus. This causes problems, such as damage to the users' hearing, when the portable audio reproducing apparatus using the earphones are listened to for an extended period of time.

### SUMMARY OF THE INVENTION

The present general inventive concept provides a noise reducing apparatus and method of minimizing background noise flowing into an earphone in a portable audio reproducing apparatus using the earphone.

The present general inventive concept also provides a portable audio reproducing apparatus to which a noise reducing apparatus and method of minimizing background noise flowing into an earphone are applied.

Additional aspects of the present general inventive concept will be set forth in part in the description which follows and, in part, will be obvious from the description, or may be learned by practice of the general inventive concept.

The foregoing and/or other aspects of the present general inventive concept may be achieved by providing a method of reducing noise in an earphone device, the method including generating an anti-noise signal being antiphase with respect to a background noise signal using a microphone having an output characteristic of being antiphase with respect to an input signal, compensating a frequency characteristic of the generated anti-noise signal, adding the compensated anti-noise signal to an audio signal to produce an added signal, and removing the background noise signal using an anti-noise component corresponding to the anti-noise signal by outputting the added signal to a speaker unit.

The foregoing and/or other aspects of the present general inventive concept may also be achieved by providing a noise reduction method, including sensing a background noise as a noise signal, receiving an audio signal and combining an inverse noise signal with the audio signal, and outputting the combined audio signal such that the noise signal of the background noise cancels the inverse noise signal when the combined audio signal is reproduced in real time.

The foregoing and/or other aspects of the present general inventive concept may also be achieved by providing a noise reduction earphone apparatus, including a microphone to sense background noise as a noise signal, a circuit unit to receive an audio signal and to combine an inverse noise signal with the audio signal, and a speaker unit to output the combined audio signal such that the noise signal of the background noise cancels the inverse noise signal when the combined audio signal is reproduced in real time.

The foregoing and/or other aspects of the present general inventive concept may also be achieved by providing a portable audio system including an earphone unit having a speaker unit and a microphone to output an audio signal and to generate anti-noise with respect to external noise, and a circuit unit to compensate a frequency characteristic of the anti-noise generated by the microphone of the earphone unit, to add the anti-noise to an input audio signal, and to remove background noise using the anti-noise by outputting the audio signal having the anti-noise to the speaker unit.

The foregoing and/or other aspects of the present general inventive concept may also be achieved by providing an audio reproducing system, including a sound source to provide an audio signal to be reproduced, and a noise reduction circuit unit to receive a detected external noise signal and to apply an inverse of the external noise signal to the audio signal before reproducing the audio signal such that the audio signal is pre-compensated for external noise occurring around the system.

The foregoing and/or other aspects of the present general inventive concept may also be achieved by providing a portable audio system including an audio reproducing unit, a noise detector, a terminal having first and second ports connected to the audio reproducing unit and the noise detector, and an external earphone device to be connected to the terminal, having a first line and a second line to be connected to the first and second ports, respectively, a speaker unit connected to the first line and a microphone unit connected to the second line, wherein the external earphone device is formed in a single monolithic body.

The foregoing and/or other aspects of the present general inventive concept may also be achieved by providing an earphone device, including a first line, a second line, a speaker unit, and a microphone unit. The first line, the second line, the speaker unit, and the microphone unit are formed in a single monolithic body.

### BRIEF DESCRIPTION OF THE DRAWINGS

These and/or other aspects of the present general inventive concept will become apparent and more readily appreciated from the following description of the embodiments, taken in conjunction with the accompanying drawings of which:

FIG. 1 illustrates an earphone used in a portable audio reproducing apparatus according to an embodiment of the present general inventive concept;

FIG. 2 illustrates waveforms exhibiting phase characteristics of output signals in relation to input signals with respect to types of microphones according to an embodiment of the present general inventive concept;

FIG. 3 illustrates a noise reduction apparatus of a portable audio reproducing apparatus according to an embodiment of the present general inventive concept;

FIG. 4 is a detailed circuit diagram illustrating a filter unit of the noise reduction apparatus of FIG. 3, according to an embodiment of the present general inventive concept;

FIG. 5 is a flowchart illustrating a method of reducing noise in an earphone apparatus according to an embodiment of the present general inventive concept;

FIG. 6 illustrates frequency characteristics having a noise reduction effect according to an embodiment of the present general inventive concept; and

FIG. 7 illustrates a portable audio reproducing apparatus according to an embodiment of the present general inventive concept.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below in order to explain the present general inventive concept by referring to the figures.

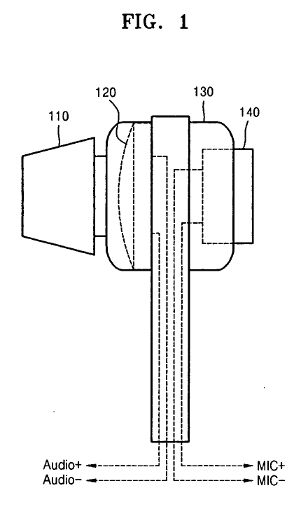

FIG. 1 illustrates an earphone used in a portable audio reproducing apparatus according to an embodiment of the present general inventive concept.

Referring to FIG. 1, the earphone includes an earpiece 110, a speaker unit 120, an earphone housing 130, and a microphone 140.

The microphone 140, which faces outside an ear, receives external noise and outputs microphone signals (MIC+, MIC−) having anti-phase output characteristics in relation to the received external noise. In other words, the microphone 140 receives an input signal (e.g., the external noise) and outputs an output signal (e.g., anti-noise) which is completely out of phase with the input signal.

The speaker unit 120, which has a rear side disposed against the microphone 140, reproduces audio signals (Audio+, Audio−) produced in the audio reproducing apparatus through the earpiece 110.

The microphone 140 may be disposed close to the speaker unit 120 and may face a direction that is opposite to a direction that the speaker unit 120 faces so that as much of the external noise can be received as possible.

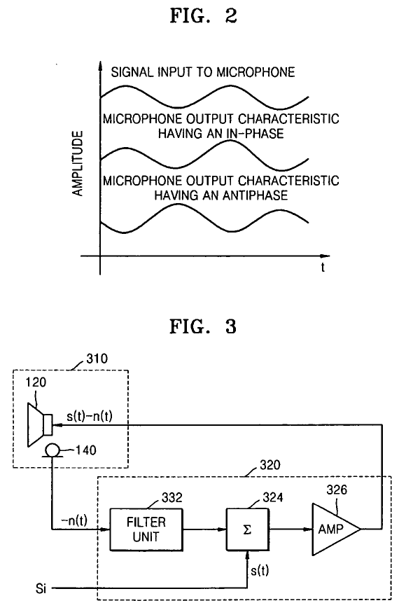

FIG. 2 illustrates waveforms exhibiting phase characteristics of output signals in relation to input signals with respect to the types of microphones according to an embodiment of the present general inventive concept.

Referring to FIG. 2, an in-phase microphone has an output characteristic of being in phase with an input signal being input. An antiphase microphone has an output characteristic of being antiphase with respect to an input signal being input.

In the present embodiment, the antiphase microphone having the output characteristic of being out of phase with (i.e., antiphase) the input signal is used. In this case, the input signal may be completely out of phase (i.e., 180 degrees out of phase) with the output signal.

FIG. 3 illustrates a noise reduction apparatus of a portable audio reproducing apparatus according to an embodiment of the present general inventive concept. It should be understood that although the embodiments of the present general inventive concept describe a portable audio reproducing apparatus, it should be understood that the embodiments of the present general inventive concept may be applied to any audio reproduction apparatus (e.g., a lap top computer, a desktop computer, a stereo, etc.) that uses headphones or earphones.

The noise reduction apparatus includes an earphone unit 310 and a noise reduction circuit unit 320. The earphone unit 310 includes a speaker unit 120 (similar to the speaker unit 120 in FIG. 1) and a microphone 140 (similar to the microphone 140 in FIG. 1). The noise reduction circuit 320 includes a filter unit 332, an addition unit 324, and an amplifier unit 326.

First, it is assumed that a background noise (i.e., external noise) of a human ear is n(t).

The microphone 140 mounted on the earphone unit 310 has an output characteristic of being antiphase with respect to (i.e., out of phase with) an input signal. The microphone 140 receives the background noise (noise signal) (n(t)) and generates an anti-noise signal (−n(t)) being antiphase with respect to the noise signal (n(t)) being input.

The filter unit 332 compensates a frequency characteristic of the anti-noise signal (−n(t)) output from the microphone 140 and performs impedance matching of an output end.

The addition unit 324 adds the anti-noise signal (−n(t)) output from the filter unit 332 and an audio signal (s(t)) input to the noise reduction circuit unit 320 from a sound source chip. That is, the addition unit 324 outputs the signal (s(t)−n(t)) obtained by adding the anti-noise signal (−n(t)) with the audio signal (s(t)). The audio signal (s(t)) is a signal having a sound set to be reproduced by the audio reproducing apparatus via the earphone unit 320 (e.g., music, voice recording, etc.).

The amplifier unit 326 amplifies a level of the signal (s(t)−n(t)) output from the addition unit 324.

The speaker unit 120 mounted on the earphone unit 310 reproduces the signal (s(t)−n(t)) output from the amplifier unit 326.

Accordingly, by minimizing a noise component corresponding to the noise signal (n(t)) existing in a sound field inside an ear using an anti-noise component corresponding to the anti-noise signal (−n(t)) generated in the speaker unit 120, the earphone unit 310 provides an optimum audio signal (s(t)).

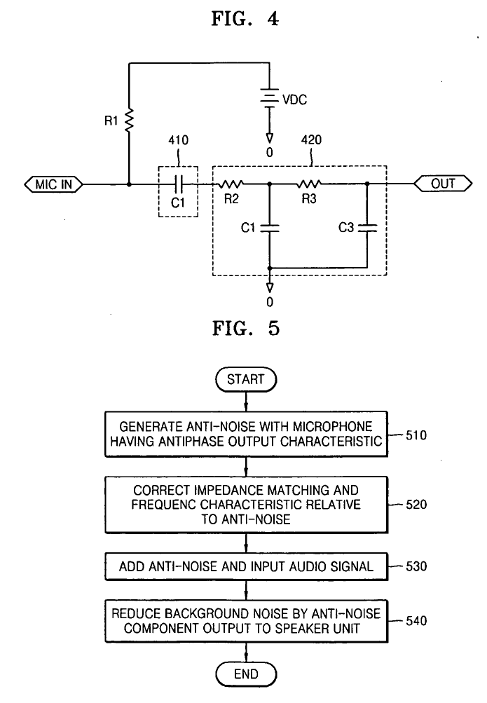

FIG. 4 is a detailed circuit diagram illustrating the filter unit 332 of FIG. 3 according to an embodiment of the present general inventive concept.

The filter unit 332 includes an impedance matching unit 410 and a low pass filter unit 420.

More specifically, referring to FIG. 4, a first resistor (R1) pulls up a signal (MIC IN) of the microphone 140 by using a voltage source (VDC).

The impedance matching unit 410 uses a first capacitor (C1). The impedance matching unit 410 removes a direct current (DC) component of the anti-noise signal (−n(t)) input through the microphone 140 while at the same time matches an impedance so that as much of a signal can be transferred to a next circuit end (i.e., from the microphone 140 to the low pass filter 420) as possible.

The low pass filter unit 420 includes a second resistor (R2), a third resistor (R3), a second capacitor (C2), and a third capacitor (C3). A frequency region in which noise can be removed is theoretically 1 KHz and below. Accordingly, by using the second and third resistors (R2, R3) and the second and third capacitors (C2, C3), the low pass filter unit 420 removes a high frequency region of the anti-noise signal output from the impedance matching unit 410, and generates an anti-noise signal in a low frequency region.

FIG. 5 is a flowchart illustrating a method of reducing noise in an earphone apparatus according to an embodiment of the present general inventive concept. The method of FIG. 5 may be performed by the noise reduction apparatus of FIG. 3.

First, an anti-noise signal is generated by a microphone having an output characteristic of being antiphase with respect to a background noise signal received in the microphone in operation 510.

Then, a frequency characteristic of the generated anti-noise signal is compensated and an impedance is matched with an output end in operation 520.

An audio signal produced by a sound source chip is added to the impedance matched anti-noise signal in operation 530 to produce an added signal.

Then, by outputting the added signal to a speaker unit, background noise existing in a sound field inside an ear is removed by an anti-noise component corresponding to the anti-noise signal in operation 540.

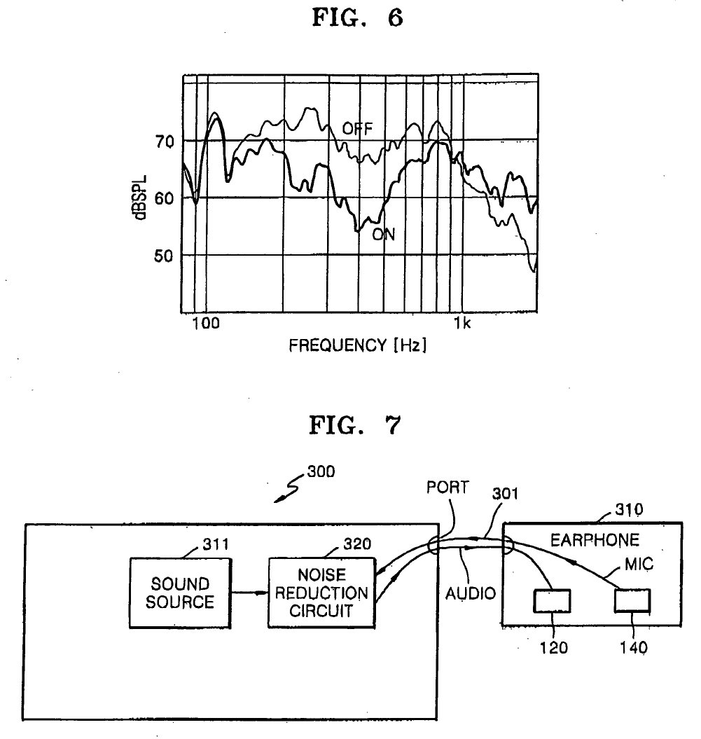

FIG. 6 illustrates frequency characteristics having a noise reduction effect according to an embodiment of the present general inventive concept.

Referring to FIG. 6, a thin solid line indicates a frequency characteristic when a noise reducing earphone is mounted and an electric circuit corresponding to the noise reduction apparatus (described above) is turned off, and a thick solid line indicates a frequency characteristic when the noise reducing earphone is mounted and the electric circuit corresponding to the noise reduction apparatus is turned on. Referring to the frequency characteristics of FIG. 6, it can be seen that when the electric circuit corresponding to the noise reduction apparatus is turned on, a sound pressure level is lowered, and the noise reduction effect is good between 300 and 500 Hz in particular.

FIG. 7 illustrates a portable audio reproducing apparatus 300 according to an embodiment of the present general inventive concept. The portable audio reproducing apparatus 300 includes a sound source unit 311 to provide an audio signal to be reproduced, the noise reduction circuit unit 320 (similar to FIG. 3), the earphone unit 310 (similar to FIG. 3), and a communication channel 301. The communication channel 301 may be a cord, wire, or a wireless communication channel. The earphone unit 310 includes the microphone 140 (similar to FIGS. 1 and 3) and the speaker unit 120 (similar to FIGS. 1 and 3). Additionally, although FIG. 7 illustrates that the noise reduction circuit unit 320 is not disposed in the earphone unit 310, it should be understood that in other embodiments of the present general inventive concept, the noise reduction circuit unit 320 may be disposed in the earphone unit 310 in communication with the microphone 140 and the speaker unit 120.

The present general inventive concept can be embodied as computer readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, and carrier waves (such as data transmission through the Internet). The computer readable recording medium can also be distributed over network coupled computer systems so that the computer readable code is stored and executed in a distributed fashion.

According to the various embodiments of the present general inventive concept as described above, by removing external noise when a portable audio reproducing apparatus using earphones or headphones is listened to, an audio signal without noise can be reproduced. Also, since a volume does not need to be set to a high level to overpower external noise, damage to a user's hearing can be prevented.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

What is claimed is:

1. A method of reducing noise in an earphone device, the method comprising:
generating an anti-noise signal being antiphase with respect to a background noise signal using a microphone having an output characteristic of being antiphase with respect to an input signal;
compensating a frequency characteristic of the generated anti-noise signal;
adding the compensated anti-noise signal to an audio signal to produce an added signal; and
removing the background noise signal using an anti-noise component corresponding to the anti-noise signal by outputting the added signal to a speaker unit.

2. The method of claim 1, wherein the compensating of the frequency characteristic comprises compensating only a low frequency component of the anti-noise signal.

3. An apparatus to reduce noise in an earphone device, the apparatus comprising:
a microphone unit to generate an anti-noise signal being antiphase with respect to a noise signal being input;
a filter unit to compensate a frequency characteristic of the anti-noise signal output from the microphone unit;
an addition unit to add the anti-noise signal output from the filter unit to an audio signal being input; and
a speaker unit to output the audio signal added in the addition unit.

4. The apparatus of claim 3, wherein the microphone unit has an output characteristic of being antiphase with respect to an input signal.

5. The apparatus of claim 3, wherein the filter unit comprises:
an impedance matching unit to match an impedance so that as much of the anti-noise signal generated by the microphone unit as possible is transferred to an output end; and
a low pass filter to pass a low frequency component of the anti-noise signal output from the impedance matching unit.

6. A noise reduction method, comprising:
sensing a background noise as a noise signal;
receiving an audio signal and combining an inverse noise signal with the audio signal; and
outputting the combined audio signal such that the noise signal of the background noise cancels the inverse noise signal when the combined audio signal is reproduced in real time.

7. A noise reduction earphone apparatus, comprising:
a microphone to sense background noise as a noise signal;
a circuit unit to receive an audio signal and to combine an inverse noise signal with the audio signal; and
a speaker unit to output the combined audio signal such that the noise signal of the background noise cancels the inverse noise signal when the combined audio signal is reproduced in real time.

8. The noise reduction earphone apparatus of claim 7, wherein the microphone provides the inverse noise signal to the circuit unit by shifting a phase of the sensed noise signal by 180 degrees.

9. The noise reduction earphone apparatus of claim 7, further comprising:
an earphone housing to house the microphone and the speaker unit such that the microphone and speaker unit face in opposite directions from each other.

10. The noise reduction earphone apparatus of claim 9, wherein a rear side of the microphone disposed against a rear side of the speaker unit.

11. The noise reduction earphone apparatus of claim 7, wherein the circuit unit comprises:
a low pass filter to pass a low frequency band of the inverse noise signal;
an addition unit to combine the inverse noise signal with the audio signal; and
an amplifier to amplify the combined audio signal and to provide the amplified audio signal to the speaker unit.

12. A portable audio system, comprising:
an earphone unit having a speaker unit and a microphone to output an audio signal and to generate anti-noise with respect to external noise; and
a circuit unit to compensate a frequency characteristic of the anti-noise generated by the microphone of the earphone unit, to add the anti-noise to an input audio signal, and to remove background noise using the anti-noise by outputting the audio signal having the added anti-noise to the speaker unit.

13. The system of claim 12, wherein the earphone unit has a microphone having an output characteristic of being antiphase with respect to an input signal.

14. The system of claim 12, wherein the speaker unit and the microphone of the earphone unit are disposed close to each other and mounted on the earphone unit.

15. An audio reproducing system, comprising:
a sound source to provide an audio signal to be reproduced; and
a noise reduction circuit unit to receive a detected external noise signal and to apply an inverse of the external noise signal to the audio signal before reproducing the audio signal such that the audio signal is pre-compensated for external noise occurring around the system.

16. The system of claim 15, wherein the inverse external noise signal is cancelled from the audio signal by the external noise when the pre-compensated audio signal is reproduced aloud.

17. The system of claim 15, further comprising:
an earphone unit to sense the external noise and generate the inverse external noise signal accordingly, and to reproduce the pre-compensated audio signal aloud.

18. A portable audio system comprising:
an audio reproducing unit;
a noise detector;
a terminal having first and second ports connected to the audio reproducing unit and the noise detector; and
an external earphone device to be connected to the terminal, having a first line and a second line to be connected to the first and second ports, respectively, a speaker unit connected to the first line and a microphone unit connected to the second line, wherein the external earphone device is formed in a single monolithic body.

19. An earphone device, comprising:
a first line;
a second line;
a speaker unit; and
a microphone unit,
wherein the first line, the second line, the speaker unit, and the microphone unit are formed in a single monolithic body.
